(19) United States
(12) Patent Application Publication
(10) Pub. No.: US 2013/0102963 A1
(43) Pub. Date: Apr. 25, 2013

(54) SECURE AUTOMATIC CONFIGURATION OF EQUIPMENT THROUGH REPLICATION

(71) Applicant: QUALCOM Incorporated, San Diego, CA (US)

(72) Inventors: Gene Wesley Marsh, Encinitas, CA (US); Rahul Anand, San Diego, CA (US); Brad L. Vaughn, San Diego, CA (US); Jen Mei Chen, San Diego, CA (US); Tao Chen, La Jolla, CA (US); Jin Guo, San Diego, CA (US); Navrina B. Singh, San Diego, CA (US); Joseph Doroba, San Diego, CA (US); Jiang Zhang, San Diego, CA (US)

(73) Assignee: QUALCOMM Incorporated, San Diego, CA (US)

(21) Appl. No.: 13/655,369

(22) Filed: Oct. 18, 2012

Related U.S. Application Data
(60) Provisional application No. 61/549,130, filed on Oct. 19, 2011.
(60) Provisional application No. 61/549,202, filed on Oct. 19, 2011.

(51) Int. Cl. G06F 15/177; A61M 5/142

(52) U.S. Cl. CPC G06F 15/177; A61M 5/142
USPC 604/151; 709/206

(57) ABSTRACT

Certain aspects of the present disclosure provide techniques and apparatus for secure automatic configuration of equipment through replication. One example method generally includes receiving a request for configuration information used to configure at least a first device to be replicated in at least a second device; and communicating the configuration information to the second device, in response to the request. At least one of the first and second wireless devices may be a medical device, and/or the configuration information may include patient treatment information. In this manner, the amount of configuration data entry by a caregiver and the number of errors in this configuration data are most likely considerably reduced. Furthermore, in an emergency, a common medical treatment may be easily configured for many patients at once (i.e., the configuration information may be easily replicated from patient device to patient device).

### CROSS-REFERENCE TO RELATED APPLICATIONS

This application claims benefit of U.S. Provisional Patent Application Ser. No. 61/549,130 (Atty. Dkt. No. 120222P1), filed Oct. 19, 2011, and U.S. Provisional Patent Application Ser. No. 61/549,202 (Atty. Dkt. No. 120222P2), filed Oct. 19, 2011, both of which are herein incorporated by reference.

### BACKGROUND

1. Field

Certain aspects of the present disclosure generally relate to wireless communications and, more particularly, to techniques for secure automatic configuration of equipment through replication.

2. Background

As various computer, electrical, and mechanical techniques have evolved, many different types of equipment have achieved increased scope and functionality to perform many new and increasingly complicated tasks. With this increased complexity, the control and configuration of such equipment are also becoming more complicated, time consuming, and potentially more error prone.

### SUMMARY

Certain aspects of the present disclosure provide an apparatus for replicating configuration information. The apparatus generally includes a receiver configured to receive a request for the configuration information used to configure the apparatus to be replicated in at least a first device, a processing system configured to operate the apparatus based on the configuration information, wherein the operation is controlled by a second device, and a transmitter configured to communicate the configuration information to the first device, in response to the request.

Certain aspects of the present disclosure provide a method for replicating configuration information. The method generally includes receiving a request for the configuration information used to configure the apparatus to be replicated in at least a first device, operating the apparatus based on the configuration information, wherein the operation is controlled by a second device, and communicating the configuration information to the first device, in response to the request.

Certain aspects of the present disclosure provide an apparatus for replicating configuration information. The apparatus generally includes means for receiving a request for the configuration information used to configure the apparatus to be replicated in at least a first device, means for operating the apparatus based on the configuration information, wherein the operation is controlled by a second device, and means for communicating the configuration information to the first device, in response to the request.

Certain aspects of the present disclosure provide a computer program product for replicating configuration information. The computer program product generally includes a computer-readable medium having instructions executable to receive a request for the configuration information used to configure the apparatus to be replicated in at least a first device; to operate the apparatus based on the configuration information, wherein the operation is controlled by a second device; and to communicate the configuration information to the first device, in response to the request.

Certain aspects of the present disclosure provide an infusion pump. The infusion pump generally includes a receiver configured to receive a request for configuration information used to configure the infusion pump to be replicated in at least a first device; a pumping mechanism for administering a solution; a processing system configured to operate the pumping mechanism based on the configuration information, wherein the operation is controlled by a second device; and a transmitter configured to communicate the configuration information to the first device, in response to the request.

Certain aspects of the present disclosure provide an apparatus for replicating configuration information. The apparatus generally includes a receiver configured to receive a request for the configuration information used to configure at least a first device to be replicated in at least a second device, a processing system configured to control at least one of the first or second device, and a transmitter configured to communicate the configuration information to the second device, in response to the request.

Certain aspects of the present disclosure provide a method for replicating configuration information. The method generally includes receiving a request for the configuration information used to configure at least a first device to be replicated in at least a second device; controlling at least one of the first or second device; and communicating the configuration information to the second device, in response to the request.

Certain aspects of the present disclosure provide an apparatus for replicating configuration information. The apparatus generally includes means for receiving a request for the configuration information used to configure at least a first device to be replicated in at least a second device, means for controlling at least one of the first or second device, and means for communicating the configuration information to the second device, in response to the request.

Certain aspects of the present disclosure provide a computer program product for replicating configuration information. The computer program product generally includes a computer-readable medium having instructions executable to receive a request for the configuration information used to configure at least a first device to be replicated in at least a second device; to control at least one of the first or second device; and to communicate the configuration information to the second device, in response to the request.

Certain aspects of the present disclosure provide a hub for replicating configuration information. The hub generally includes at least one antenna; a receiver configured to receive, via the at least one antenna, a request for the configuration information used to configure at least a first device to be replicated in at least a second device; a processing system configured to control at least one of the first or second device; and a transmitter configured to communicate, via the at least one antenna, the configuration information to the second device, in response to the request.

Certain aspects of the present disclosure provide a first apparatus for replicating configuration information. The apparatus generally includes a receiver configured to receive, from a second apparatus, the configuration information used by the second apparatus to configure at least one first device; and a processing system configured to control at least one second device based on the configuration information.

Certain aspects of the present disclosure provide a method for replicating configuration information at a first apparatus. The method generally includes receiving, from a second apparatus, the configuration information used by the second apparatus to configure at least one first device; and controlling at least one second device based on the configuration information.

Certain aspects of the present disclosure provide an apparatus for replicating configuration information. The apparatus generally includes means for receiving a request for the configuration information used to configure at least a first device to be replicated in at least a second device, means for controlling at least one of the first or second device, and means for communicating the configuration information to the second device, in response to the request.

Certain aspects of the present disclosure provide a computer program product for replicating configuration information at a first apparatus. The computer program product generally includes a computer-readable medium having instructions executable to receive, from a second apparatus, the configuration information used by the second apparatus to configure at least one first device; and to control at least one second device based on the configuration information.

Certain aspects of the present disclosure provide a hub for replicating configuration information. The hub generally includes at least one antenna; a receiver configured to receive, via the at least one antenna from an apparatus, the configuration information used by the apparatus to configure at least one first device; and a processing system configured to control at least one second device based on the configuration information.

Certain aspects of the present disclosure provide a system for replicating the programming of a first device into a second device capable of receiving that configuration. The configuration may be transferred over a wireless (or a wired) communication link. For certain aspects, the configuration may be broadcast to multiple new devices simultaneously. The first and second devices may be used in medical applications. According to certain aspects, the transfer may occur through a third wireless device, which may request the configuration from the first device and pass it on to the second device (e.g., the replicating device). In this case, the third device may be a controller for both the first and second devices. The third device may offer the opportunity to adjust the retrieved configuration before programming the designated device.

Certain aspects of the present disclosure provide a system for transferring control of a set of devices from a source controller to a target controller, wherein the source controller replicates its configuration data to the target controller, and then commands its controlled devices to transfer their communication links to the target controller.

Certain aspects of the present disclosure provide a system for transferring control of a set of devices from a source controller to a target controller, wherein the source controller's devices each individually replicate their configuration data to the target controller.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 illustrates a diagram of an example communications network in accordance with certain aspects of the present disclosure.

FIG. 2 illustrates a block diagram of an example wireless device in accordance with certain aspects of the present disclosure.

FIG. 3 illustrates an example system for transferring configuration information from a source device to a target device in accordance with certain aspects of the present disclosure.

FIG. 4 illustrates an example system for transferring control of devices associated with a source control hub to a target control hub, in accordance with certain aspects of the present disclosure.

FIG. 5 illustrates an example system for transferring configuration of devices associated with a source control hub to corresponding devices associated with a target control hub, in accordance with certain aspects of the present disclosure.

FIGS. 6-8 illustrate example operations for replicating configuration information in accordance with certain aspects of the present disclosure.

FIGS. 6A-8A illustrate example means for performing the operations shown in FIGS. 6-8.

### DETAILED DESCRIPTION

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different wireless technologies, system configurations, networks, and transmission protocols, some of which are illustrated by way of example in the figures and in the following description of the preferred aspects. The detailed description and drawings are merely illustrative of the disclosure rather than limiting, the scope of the disclosure being defined by the appended claims and equivalents thereof.

### An Example Communication System

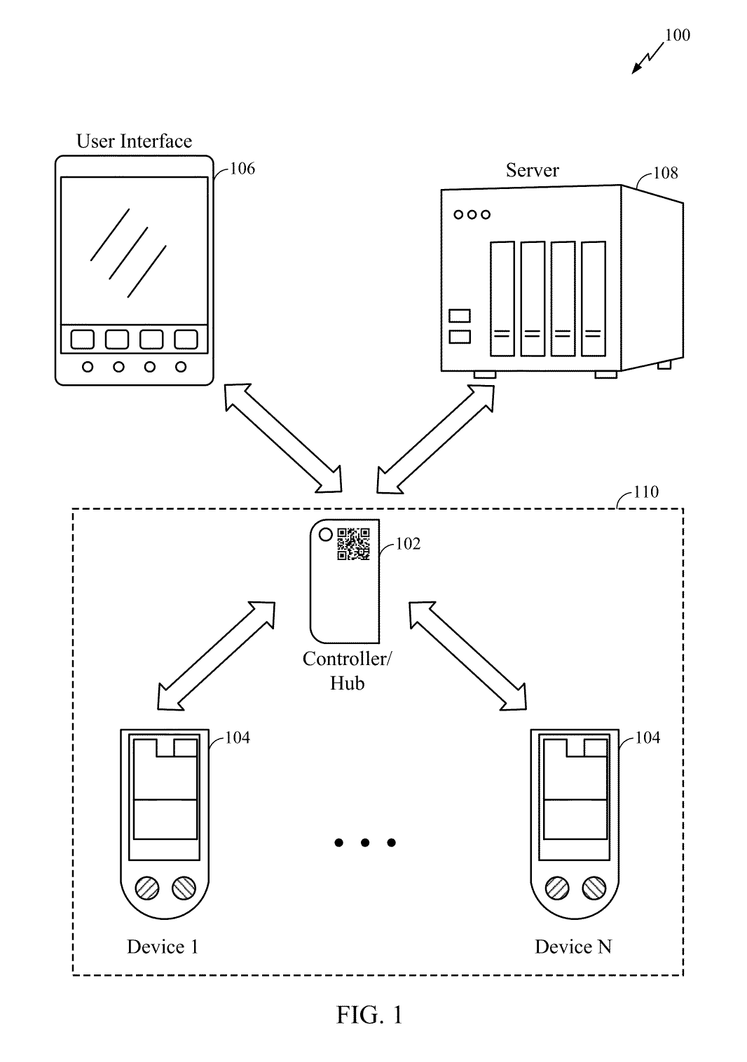

FIG. 1 illustrates an example distributed communication system 100 with a controller/hub 102 and one or more wireless devices 104. The controller/hub 102 may communicate with the wireless devices 104 via one or more wireless channels using one or more antennas and/or via one or more wired connections. The controller/hub 102 may function similar to an access point (AP) or a Wi-Fi hotspot in an IEEE 802.11 network.

The communication system 100 may be used in a healthcare environment, such as a hospital, clinic, hospice, or home. In such cases, the wireless devices 104 may include any of various suitable wireless medical devices, such as an infusion pump, a blood pressure monitor, a pulse oximeter, an electrocardiograph (ECG), and the like. The controller/hub 102 may be associated with a single patient, and the controller/hub 102 and the wireless devices associated therewith may form an in-room network 110. The in-room network 110 may function similar to a local area network (LAN) or a home network. Certain wireless devices 104 may be worn by the patient (e.g., a finger-worn or wrist-worn unit); inserted or implanted into the patient's body; or attached to or embedded in the patient's bed, gurney, clothing, or other devices that would generally stay physically close to the patient (e.g., a walker, cane, watch, or glasses).

Wireless communication between the controller/hub 102 and a wireless device 104 may use any of various suitable wireless technologies, such as near field communication (NFC), Bluetooth, Bluetooth Low Energy (BTLE), Wi-Fi in accordance with the IEEE 802.11 standard, Zigbee, ANT/ANT+, Toumaz Healthcare Ltd.'s Sensium platform for developing body area networks (BANs), Medical Implant Communication Service (MICS), and the like. The wireless communication may also occur via infrared (IR), bar code scanning, or other optical technologies.

A user interface 106 may provide an interface for a user (e.g., a doctor, a nurse, or the patient himself) to communicate with the controller/hub 102. The user interface 106 may comprise a networked device, such as a tablet (as shown), a smart phone, a cellular phone, a laptop, or dedicated electronic hardware. The user interface 106 may be connected with the controller/hub 102, a healthcare facility intranet (e.g., a hospital intranet), or the wireless device 104 directly via physical wires, wirelessly, or both. The connection between the user interface 106 and the controller/hub 102 or the wireless device 104 may be part of the in-room network 110.

A server 108 may provide an interface between the controller/hub 102 and a healthcare facility intranet. As part of the healthcare information system (HIS), the server 108 may store and provide access to electronic medical records (EMRs) of the patients and may provide the intelligence for checking therapies against patient allergies, preventing conflicting medications, etc. For certain aspects, the controller/hub 102 may provide the only interface between the in-room network 110 and the facility intranet. The controller/hub 102 may be connected with the facility intranet directly via a wired technology (e.g., Ethernet), indirectly via a wireless router connected to the server 108 via a wired technology, or indirectly via a public or private wired, wireless, or hybrid network technology.

One example scenario of the interactions between the various apparatus in the communication system 100 in a healthcare environment involves drug delivery to a patient. First, an infusion pump (e.g., a wireless device 104) may scan the patient's identification (ID), which may be contained in a bar code wristband worn by the patient. Then NFC may be used for out-of-band pairing between the controller/hub 102 and the pump. The pump may inform the controller/hub 102 of the patient's ID. Based on this, the controller/hub 102 may query the HIS/EMR for the patient's treatment information. Once this information is received, the controller/hub 102 may configure the pump (i.e., may transmit configuration information to the pump) for a particular intravenous (IV) therapy treatment. The configuration information may include the set of solution and medication, the solution's density, the flow rate, the total volume to infuse, and an interval (in an intermittent flow pattern) for an intravenous therapy the patient is prescribed to receive. The user interface 106 may prompt the caregiver to confirm the treatment, and once the caregiver confirms, the caregiver may scan a bar code, a quick response (QR) code, or a radio frequency identification (RFID), for example, on an IV solution bag before or after the caregiver connects the bag with the pump. The controller/hub 102 may signal the infusion pump to begin the treatment, perhaps at the command of the caregiver. Data from the pump may be transmitted to the controller/hub 102, and particular received data may be transmitted to the HIS via the healthcare facility intranet or other wireless or wired links.

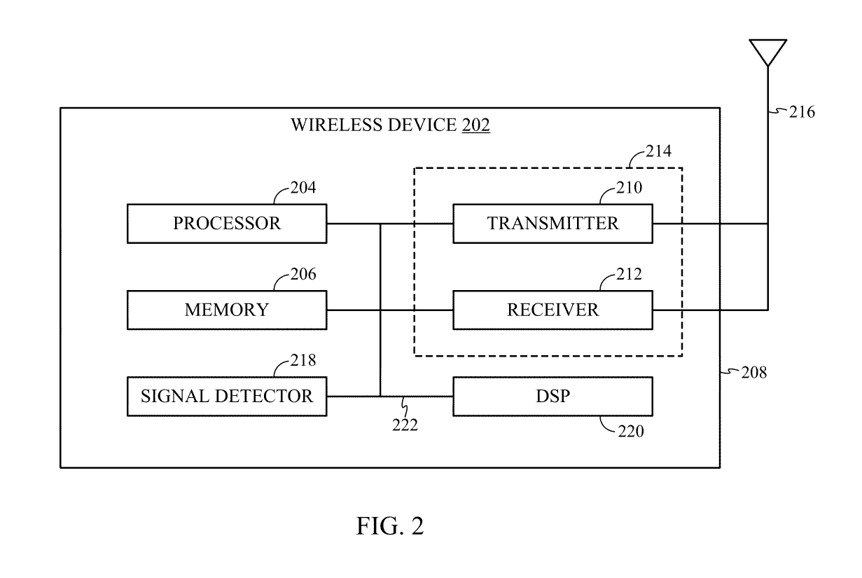

FIG. 2 illustrates various components that may be utilized in a wireless device 202. The wireless device 202 is an example of a device that may be configured to implement the various methods described herein. The wireless device 202 may be a controller/hub 102 or a wireless device 104, as described above with respect to FIG. 1.

The wireless device 202 may include a processor 204 which controls operation of the wireless device 202. The processor 204 may also be referred to as a central processing unit (CPU). Memory 206, which may include both read-only memory (ROM) and random access memory (RAM), provides instructions and data to the processor 204. A portion of the memory 206 may also include non-volatile random access memory (NVRAM). The processor 204 typically performs logical and arithmetic operations based on program instructions stored within the memory 206. The instructions in the memory 206 may be executable to implement the methods described herein.

The wireless device 202 may also include a housing 208 that may include a transmitter 210 and a receiver 212 to allow transmission and reception of data between the wireless device 202 and a remote location. The transmitter 210 and receiver 212 may be combined into a transceiver 214. An antenna 216 may be attached to the housing 208 and electrically coupled to the transceiver 214. The wireless device 202 may also include (not shown) multiple transmitters, multiple receivers, multiple transceivers, and/or multiple antennas.

The wireless device 202 may also include a signal detector 218 that may be used in an effort to detect and quantify the level of signals received by the transceiver 214. The signal detector 218 may detect such signals as total energy, pilot energy from pilot subcarriers or signal energy from the preamble symbol, power spectral density, and other signals. The wireless device 202 may also include a digital signal processor (DSP) 220 for use in processing signals.

The various components of the wireless device 202 may be coupled together by a bus system 222, which may include a power bus, a control signal bus, and a status signal bus in addition to a data bus.

### Example Secure Automatic Configuration of Equipment Through Replication

Medical equipment is becoming increasingly computerized. While this provides many benefits, it also frequently involves cumbersome configuration procedures. In many cases, a new piece of medical equipment, for example, may be replacing another that has insufficient battery charge, is no longer considered sterile, has sustained damages, etc. Typically, when a new piece of equipment replaces an already configured piece of equipment, the new equipment is configured in the same manner as the original equipment was configured. The configuration process may most likely be repeated for every change of the equipment. For instance, a patient may be continually given a particular intravenous (IV) infusion via a pump. As one round of treatment ends, another bag is brought in, and the complete configuration of the original treatment is normally repeated.

In certain aspects, if the two pieces of equipment possess the capability to communicate with each other, it is possible to pass on configuration information from the old equipment to the new equipment, thereby easing the configuration burden. In certain aspects, if a replicating device is able to communicate with the devices whose functionality it is replicating, directly or via a third (wired or wireless) device, then a protocol may be established whereby the replacing device may receive the configuration of the device it is replacing, suitably verified and optionally modified. For certain aspects, the third device may be a controller of the two devices. This replication considerably reduces the amount of configuration data entry inputted by a caregiver and may most likely result in a reduction of errors in those configurations. Also, in an emergency, such as an epidemic outbreak of a contagious disease or after a natural disaster, a common medical treatment may easily be configured for many people at once. Furthermore, all devices in a system may be handed over from one controller to another using this configuration capability.

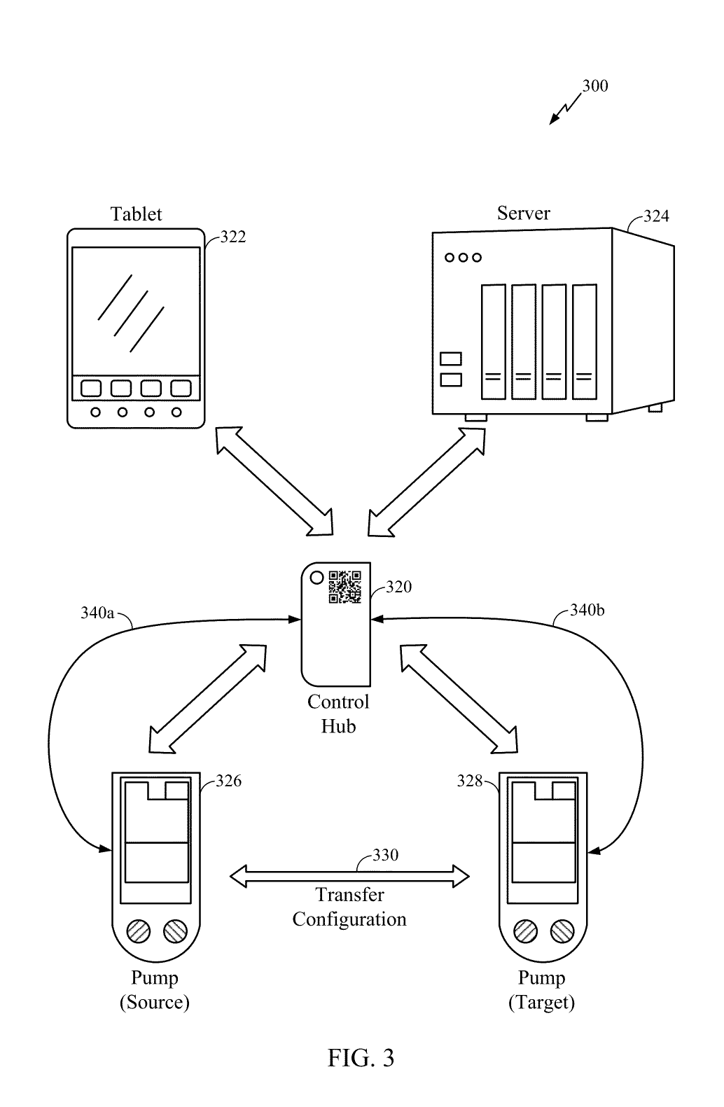

FIG. 3 illustrates an example system 300 for transferring configuration information from a source device (such as an infusion pump) to a target device in accordance with certain aspects of the present disclosure. The system 300 includes a tablet 322, a server 324, a source pump 326, and a target pump 328 communicatively coupled to a control hub 320 (e.g., controller/hub 102 shown in FIG. 1).

In certain aspects, the source and target pumps 326, 328 and the control hub 320 are part of an in-room network 110 typically associated with a single patient. The control hub 320 typically interfaces with the tablet 322 and the server 324 and controls all other in-room equipment, such as the pumps 326, 328 a blood pressure monitor, a pulse oximeter, an electrocardiograph (ECG), or a patient's wrist-worn unit. The control hub 320 may control the in-room equipment through updating the configuration information or setting or adjusting one or more particular operational parameters. The control hub 320 may be communicatively coupled to the other in-room equipment, the tablet 322, and the server 324 via wired or wireless links and may provide access to the other in-room equipment through a user interface device, such as the tablet 322.

The server 324 typically provides an interface between the control hub 320 and a healthcare network (e.g., a hospital network) (not shown); provides access to information such as work lists, patient records, information about medicines to be delivered, etc.; and relays status reports from the control hub 320. The tablet 322 provides a user interface for the system 300. The tablet 322 may be used by a caregiver (e.g., a nurse, physician's assistant (PA), doctor, etc. in a hospital or clinic, for example) to access information on the server 324 and the healthcare network via the server 324. The tablet 322 may also be used by the caregiver to issue commands to the control hub 320 and the medical equipment via the control hub 320, and to view status of the network and the equipment. Pumps 326, 328 are used for medication delivery and typically control a rate of flow of medication to a patient. A pump 326, 328 may be associated with a particular intravenous (IV) bag, for example, identified by a unique bar code which may be scanned by the pump. The in-room equipment may further include patient-worn sensors such as an electrocardiograph (ECG), a blood pressure monitoring device, a pulse oximeter, a thermometer, and the like.

In certain aspects, the system enables transferring configuration information between devices of the in-room network 110 directly or via the control hub. For example, a caregiver wishing to duplicate the programming of a source pump 326 in a target pump 328 without using the control hub 320 may begin by optionally authenticating himself/herself to each of the pumps. This authentication may be accomplished, for example, using an electronic credential such as a user device equipped with a certificate, or a user name and password pair, etc. The caregiver may then associate the target pump 328 with the source pump 326 using a secure communication channel 330. The source pump 326 may then autonomously or at the caregiver's command transfer its configuration data to the target pump 328 via the communication channel 330.

In certain aspects, the configuration data may be encrypted and/or protected with a secure checksum in an effort to ensure integrity of the data. The configuration data may, for example, include parameters desired for control of the source pump 326, a current status of its operation, data logged for record keeping or performance tracking, progress of a treatment administered by the pump, etc. In an aspect, the caregiver may be given an opportunity to modify the configuration data at the source pump 326 before the transfer or at the target pump 328 after the transfer. The caregiver may then decommission the source pump 326 and commence the operation of the target pump 328 (in either order or simultaneously), possibly continuing the operation from where the source pump 326 left off. In this configuration, the source and/or the target pumps may be virtual devices, as represented, for example, in the database of a device controller.

In certain aspects, a caregiver wishing to duplicate the programming of the source pump via the control hub 320 would begin by authenticating himself/herself to the control hub 320. This authentication may be accomplished using an electronic credential such as a user device equipped with a certificate, or a user name and password pair, or the like. The caregiver may then associate the target pump 328 with the control hub 320. After selecting the source pump 326, the control hub 320 may be commanded to perform the replication. The control hub 320 may fetch the configuration of the source pump 326, either by querying it over an encrypted channel 340a, or from a cache, and then present that configuration to the caregiver (e.g., on the tablet 322). The configuration may, for example, include parameters desired for control of the source pump 326, the current status of its operation, data logged for performance tracking, progress of a treatment, etc. The caregiver may be given an opportunity to modify the configuration data using the tablet 322, for example, before commanding the control hub 320 to transfer the configuration data. The caregiver may then select the target pump 328 for the transfer. The control hub 320 may establish a secure link 340b to the target pump 328 and then transfer the configuration information to the target pump 328 via the link 340b. In an aspect, once the target pump 328 is successfully configured, the source pump 326 may be decommissioned, and operation of the target pump 328 may then begin, possibly continuing the operation from where the source device left off.

In certain aspects, the caregiver may select more than one target device for the transfer, which may be already associated devices or newly associated devices. In an aspect, a secure broadcast or multicast link may be established to multiple target devices, and the configuration data may be transferred in parallel to these target devices.

The mechanisms described above may be combined to enable other applications. For instance, one may propagate a particular configuration across an extended network of devices. As another example, one may manage the transfer of devices between two controllers. In this latter example, one of the two controllers may be active and has a number of devices associated with it, and the other of the two controllers has not yet been configured and has no devices. The device currently designated as the controller may be referred to as the source, and the other controller may be designated as the target. Transfer of configuration data using the two-device implementation above may likely provide all of the data specified for the target controller to assume the duties of the source controller. An additional operation may be initiated that transfers the secure link information for the target controller into the devices managed by the source controller, resulting in a smooth transfer of control. In another aspect, each device managed by the source controller may individually be re-associated with the target controller, such action triggering a transfer in the two-device implementation between the new device and the target controller. Once all devices are paired, the source controller may be safely decommissioned.

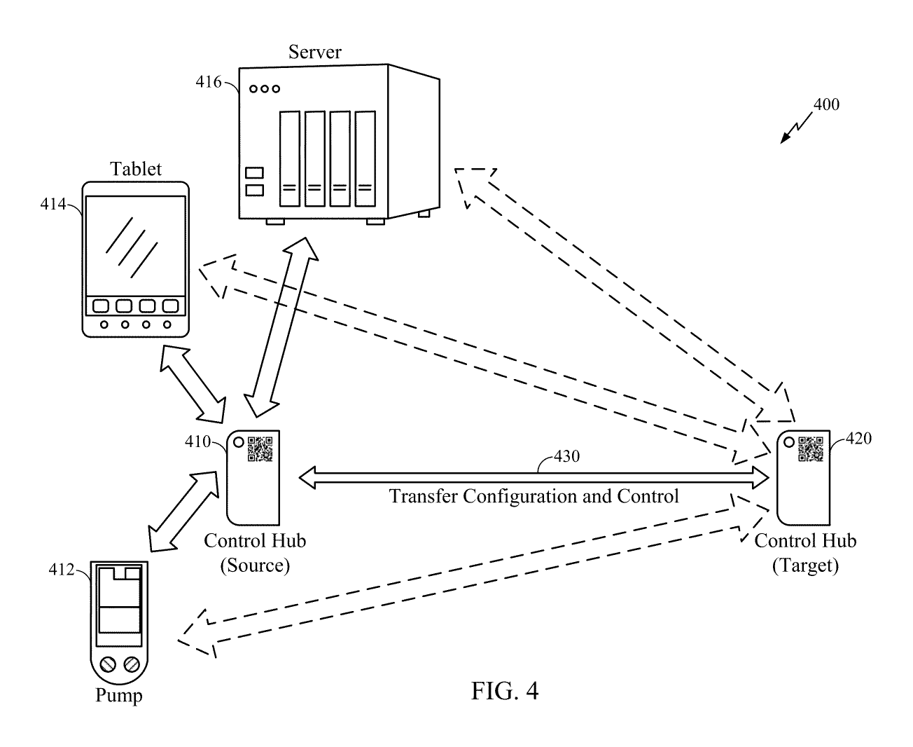

FIG. 4 illustrates an example system 400 for transferring control of devices associated to a source control hub to a target control hub in accordance with certain aspects of the present disclosure. System 400 includes a source control hub 410 that controls the operation of an in-room network including a pump 412 and interfaces with a tablet 414 and a server 416. In certain aspects, the source control hub 410 may be replaced with a target control hub 420, and the control of the in-room devices may be transferred to the target control hub 420. A caregiver wishing to carry out such a transfer may first associate the target control hub 420 to the source control hub 410 via a secure communication link 430. The user may then command the source control hub 410 to transfer the configuration and control of the in-room devices to the target control hub 420. The source control hub 410 may fetch the configuration of the in-room devices (e.g., pump 412) and transfer the same to the target control hub 420 over the secure communication channel 430. Alternatively, the source control hub may transfer the configuration of the in-room devices from a local cache. Additionally, the source control hub 410 may transfer the control of the in-room devices to the target control hub 420. This may include transferring link information associated with the in-room devices. In certain aspects, each in-room device may individually be re-associated with the target control hub 420. Once, all the in-room devices are paired with the target control hub 420 and the target control hub 420 successfully assumes the responsibility of the source control hub 410, the source control hub 410 may be safely decommissioned.

In certain aspects, certain medical devices, such as pumps (and sometimes control hubs), are not considered sufficiently sterile after operating for a particular number of days. In such cases, old pumps and/or an associated control hub may be replaced with new corresponding pumps and/or a new control hub. The configuration of the old devices may thus be transferred to the corresponding new devices associated to the new control hub for seamless operation.

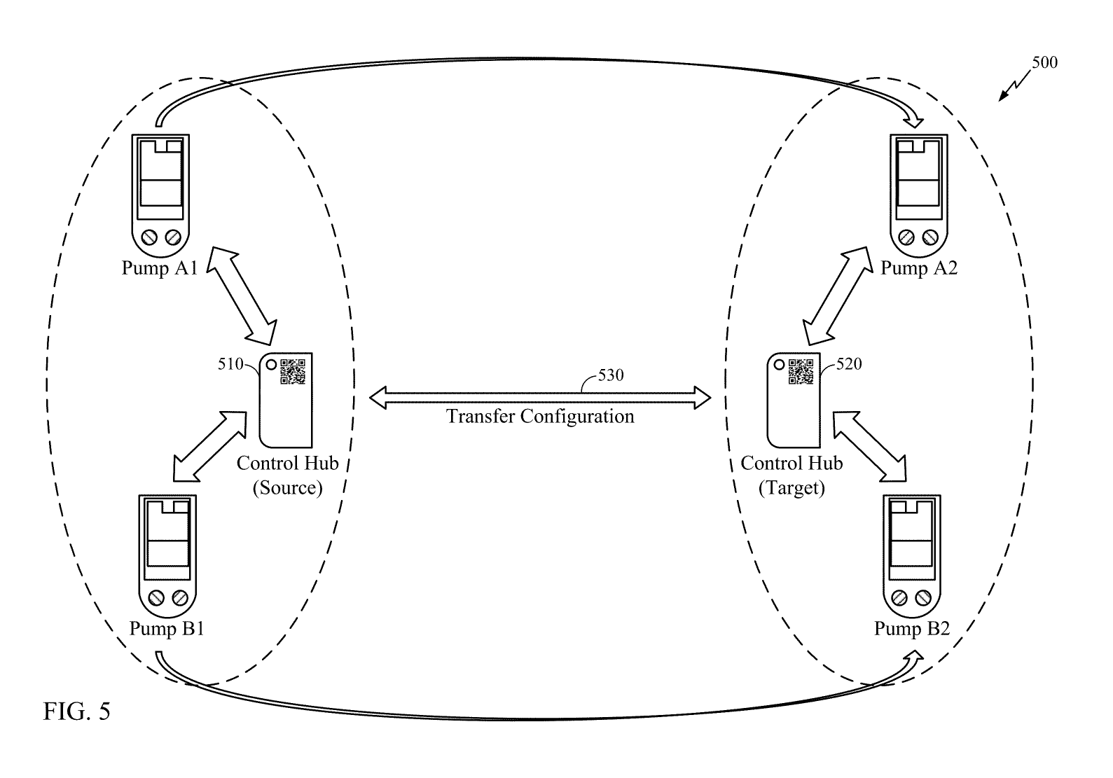

FIG. 5 illustrates an example system 500 for transferring configuration of devices associated with a source control hub to corresponding devices associated with a target control hub in accordance with certain aspects of the present disclosure. The system 500 includes a source control hub 510 that controls the operation of an in-room network including pumps A1 and B1. A caregiver wishing to replace the source control hub 510 and associated pumps A1 and B1 with a new control hub and corresponding new pumps, may first associate new pumps A2 (corresponding to A1) and B2 (corresponding to B1) to a new target control hub 520. The caregiver may then establish a secure communication link 530 between the source control hub 510 and the target control hub 520 and command the source control hub 510 to transfer configuration of the pumps A1 and B1 to the target control hub 520. The source control hub 510 may fetch the configuration of the pumps A1 and B1, and transfer the same to the target control hub 520 over the secure communication link 530. Alternatively, the source control hub 510 may transfer the configuration of the pumps A1 and B1 from a local cache. The caregiver may then command the target control hub 520 to configure pump A2 with the configuration of pump A1 and configure pump B2 with the configuration of pump B1. Once, the pumps A2 and B2 are configured and ready to operate, the pumps A1 and B1 as well as the source control hub 510 may be decommissioned, and the target control hub 520 and pumps A2 and B2 may take over.

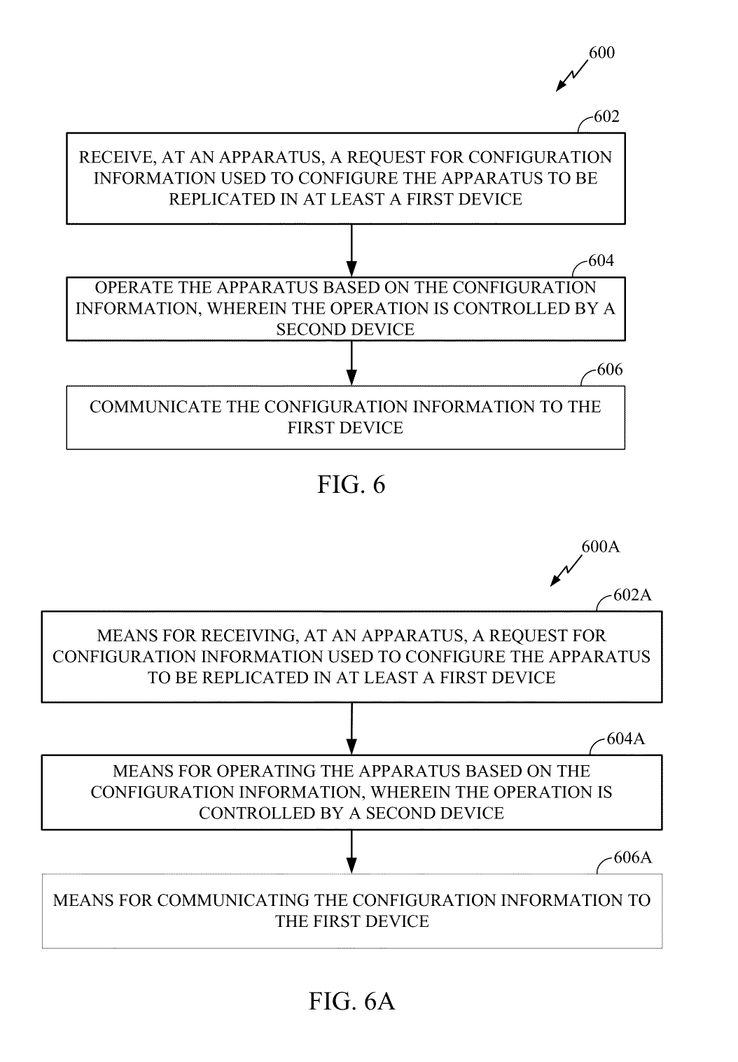

FIG. 6 illustrates example operations 600 for replicating configuration information in accordance with certain aspects of the present disclosure. The operations 600 may be performed by an apparatus, such as a wireless device 104. The operations 600 may start, at 602, with the apparatus receiving a request for configuration information used to configure the apparatus to be replicated in at least a first (wireless-capable) device. The configuration information may include patient treatment information. At 604, the apparatus may be operated based on the configuration information, and this operation may be controlled by a second (wireless-capable) device (e.g., a controller/hub 102). At 606, the apparatus may communicate the configuration information to the first device, in response to the request received at 602.

According to certain aspects, the apparatus may determine whether to continue or stop the operation of the apparatus after receiving the request at 602. For certain aspects, the apparatus may cease the operation of the apparatus after the configuration information had been communicated at 606. For certain aspects, at least one of starting of the operation, stopping of the operation, monitoring progress of the operation, or managing an alarm based on the operation is controlled by the second device.

For certain aspects, the communicating at 606 includes transferring the configuration information to the first device via the second device. For certain aspects, the request is received from the second device, and the communicating comprises transmitting the configuration information to the second device, for transferring to the first device. The second device may provide an opportunity to adjust configuration information prior to transfer of the configuration information to the first device and, if the configuration information is adjusted, may communicate the adjusted configuration information to the first device. For certain aspects, the second device may control the apparatus and the first device. For certain aspects, the communicating may entail wirelessly transmitting the configuration information to the first device and one or more additional (wireless-capable) devices. The communicating may involve transferring the configuration information wirelessly for certain aspects.

According to certain aspects, the apparatus may adjust the configuration information after receiving the request at 602, but prior to communicating the configuration information to the first device at 606. For certain aspects, the configuration information is adjusted based on progress of a treatment administered thus far by the apparatus. For example, if a patient is to receive 1 L of a solution, and the apparatus has already administered ⅔ L, the configuration information may be adjusted to indicate only ⅓ L remains to be administered. For other aspects, the second device may adjust the configuration information after receipt. Such adjustment to a previously valid configuration may be made to perform the next phase of a treatment procedure, which may entail a different configuration than the previous phase.

According to certain aspects, at least one of the apparatus or the first device is a medical device, such as an infusion pump. In this case, the configuration information may include at least one of parameters for controlling the apparatus, a current status of operation of the apparatus, data logged in the apparatus for record keeping or performance tracking, or progress of a treatment administered by the apparatus which is optionally used for the next device (e.g., the first device) to continue a partially delivered treatment, or other cumulative records in the apparatus that may be appended to by the next device.

According to certain aspects, the apparatus may receive the request at 602 based on at least one of: a treatment provided by the apparatus has nearly or completely run out; the apparatus is no longer considered sterile; the apparatus has or nearly has insufficient battery charge; or the apparatus has sustained damage. For certain aspects, the apparatus may receive the request after the request is initiated or approved by another apparatus, such as a user interface 106.

According to certain aspects, the operations 600 may further include locally storing the configuration information in a memory of the apparatus. In this case, the apparatus may retrieve the configuration information from the memory in response to the request at 602.

For certain aspects, the request may be expressed by physically locating the apparatus and the first device close to each other such that a short range radio or a proximity sensor detects in one (or both of the devices can detect) the presence of the counterpart device (i.e., the other device). For certain aspects, the request is received if the apparatus is physically located close (e.g., within 1 m, or in some cases such as NFC, within 5 cm) to the first device. For certain aspects, the request is received only after the request is first initiated or approved by a user interface 106.

According to certain aspects, the request is received at 602 due to an emergency, such as a natural disaster or an epidemic. For other aspects, the request is received based on at least one of a treatment provided by the apparatus has nearly or completely run out; the apparatus is no longer considered sterile; the apparatus has or nearly has insufficient battery charge; or the apparatus has sustained damage.

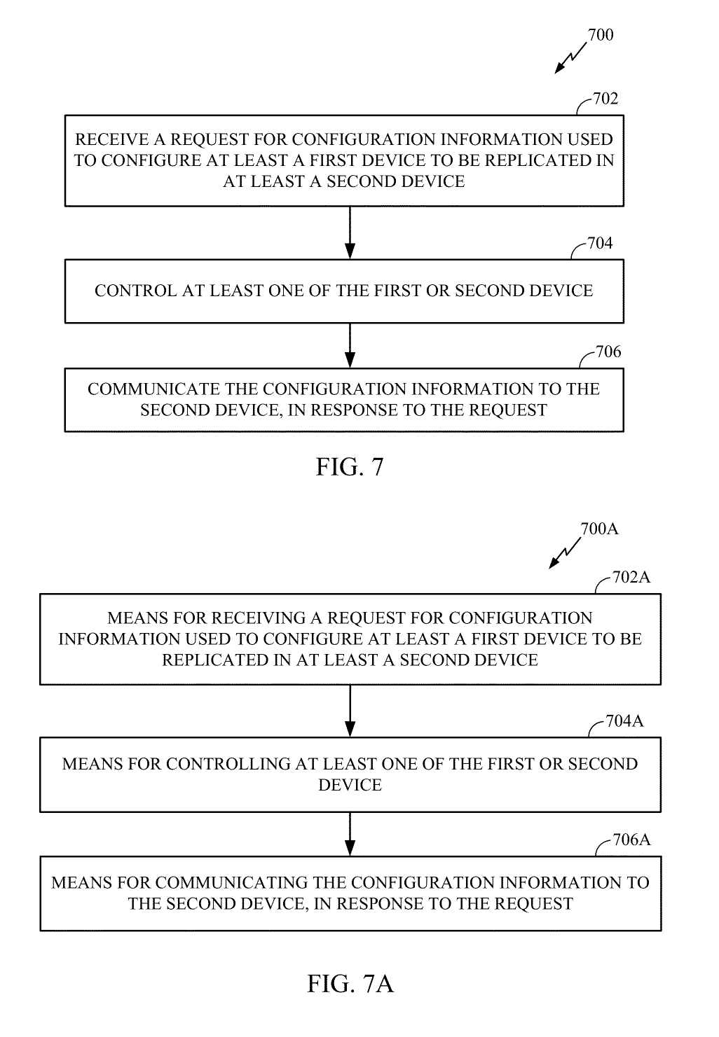

FIG. 7 illustrates example operations 700 for replicating configuration information in accordance with certain aspects of the present disclosure. The operations 700 may be performed by an apparatus, such as a controller/hub 102. The operations 700 may begin, at 702, with the apparatus receiving a request for configuration information used to configure at least a first (wireless-capable) device to be replicated in at least a second (wireless-capable) device. For certain aspects, the configuration information includes patient treatment information. At least one of the first or second device may be a medical device, such as an infusion pump. As described above, the configuration information may include at least one of parameters for controlling the first device, a current status of operation of the first device, data logged in the first device for record keeping or performance tracking, or progress of a treatment administered by the first device which is optionally used for the next device (e.g., the second device) to continue a partially delivered treatment, or other cumulative records in the first device that may be appended to by the next device.

At 704, the apparatus may control at least one of the first or second device. According to certain aspects, the apparatus may control the at least one of the first or second device by at least one of starting an operation based on the configuration information, stopping the operation, monitoring progress of the operation, or managing an alarm based on the operation.

At 706, the apparatus may communicate the configuration information to the second device, in response to the request. For certain aspects, the apparatus may adjust the configuration information before communicating with the second device at 706.

According to certain aspects, the operations 700 may further include the apparatus retrieving the configuration information from another apparatus in response to the request. The other apparatus may be a server 108, for example.

According to certain aspects, the apparatus may communicate the configuration information at 706 by transferring the configuration information to the second device via a third (wireless-capable) device. In such cases, the apparatus may control a first set of (wireless-capable) devices that includes the first device; the third device may control a second set of (wireless-capable) devices that includes the second device; and the apparatus may communicate the configuration information for the first set of devices to be replicated in the second set of devices. The third device may be a hub, such as a controller/hub 102.

According to certain aspects, the apparatus may receive the configuration information from the first device. In such cases, the apparatus may obtain, from at least one of another apparatus (e.g., a server 108) or a user interface associated with the apparatus, for example, at least one of adjustment information for (or confirmation of) the configuration information received from the first device, before communicating the configuration information to the second device at 706. If the adjustment information is obtained, the apparatus may adjust the configuration information based on the adjustment information before the communicating at 706. The communicating at 706 may include communicating the adjusted configuration information to the second device.

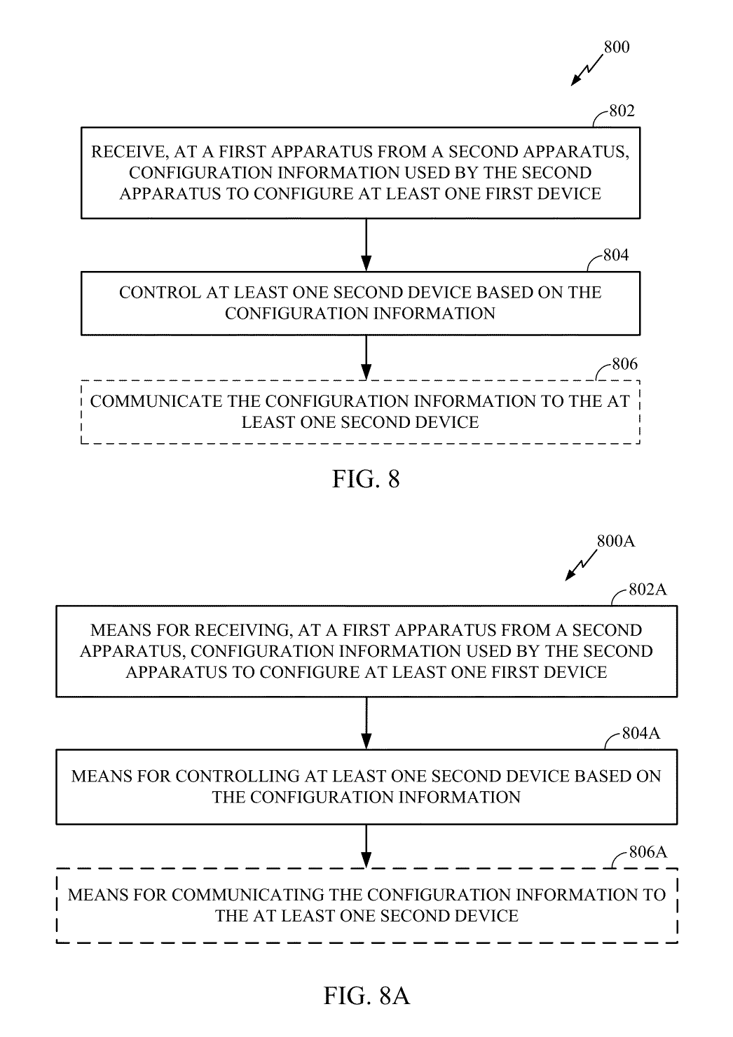

FIG. 8 illustrates example operations 800 for replicating configuration information in accordance with certain aspects of the present disclosure. The operations 800 may be performed by a first apparatus, such as a controller/hub 102. The operations 800 may begin, at 802, with the first apparatus receiving, from a second apparatus, configuration information used by the second apparatus to configure at least one first (wireless-capable) device.

At 804, the first apparatus may control at least one second (wireless-capable) device based on the configuration information. For certain aspects, the at least one second device is the at least one first device. For certain aspects, the first apparatus may control the at least one second device at 804 by at least one of starting an operation based on the configuration information, stopping the operation, monitoring progress of the operation, or managing an alarm based on the operation.

As described above, the configuration information may include at least one of parameters for controlling the first device, a current status of operation of the first device, data logged in the first device for record keeping or performance tracking, or progress of a treatment administered by the first device which is optionally used for the next device (e.g., the second device) to continue a partially delivered treatment, or other cumulative records in the first device that may be appended to by the next device.

According to certain aspects, the first apparatus may communicate the configuration information to the at least one second device at 806. In such cases, the first apparatus may adjust the configuration information before communicating with the at least one second device at 806. In other such cases, the first apparatus may obtain, from at least one of a third apparatus (e.g., a server 108) or a user interface associated with the first apparatus, for example, at least one of adjustment information for (or confirmation of) the configuration information received from the second apparatus, before communicating the configuration information to the at least one second device at 806. The communicating at 806 may include communicating the adjusted configuration information to the at least one second device.

The various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering. For example, operations 600 illustrated in FIG. 6 correspond to means 600A illustrated in FIG. 6A.

For example, means for transmitting or means for communicating may comprise a transmitter, such as the transmitter 210 of the wireless device 202 illustrated in FIG. 2. Means for receiving or means for communicating may comprise a receiver, such as the receiver 212 of the wireless device 202 shown in FIG. 2. Means for communicating, means for retrieving, means for obtaining, means for adjusting, means for operating, means for controlling, means for determining, and/or means for processing may comprise a processing system, which may include one or more processors, such as processor 204 illustrated in FIG. 2. Means for storing and/or means for retrieving may comprise a storage medium, such as memory 206 depicted in FIG. 2.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory), and the like. Also, “determining” may include resolving, selecting, choosing, establishing, and the like.

As used herein, a phrase referring to “at least one of” a list of items refers to any combination of those items, including single members. As an example, “at least one of a, b, or c” is intended to cover: a, b, c, a-b, a-c, b-c, and a-b-c.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the present disclosure may be embodied directly in hardware, in a software module executed by a processor, or in a combination of the two. A software module may reside in any form of storage medium that is known in the art. Some examples of storage media that may be used include random access memory (RAM), read only memory (ROM), flash memory, EPROM memory, EEPROM memory, registers, a hard disk, a removable disk, a CD-ROM, and so forth. A software module may comprise a single instruction, or many instructions, and may be distributed over several different code segments, among different programs, and across multiple storage media. A storage medium may be coupled to a processor such that the processor can read information from, and write information to, the storage medium. In the alternative, the storage medium may be integral to the processor.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a wireless node. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement the signal processing functions of the PHY layer. In the case of a user terminal or other wireless node, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processor may be responsible for managing the bus and general processing, including the execution of software stored on the machine-readable media. The processor may be implemented with one or more general-purpose and/or special-purpose processors. Examples include microprocessors, microcontrollers, DSP processors, and other circuitry that can execute software. Software shall be construed broadly to mean instructions, data, or any combination thereof, whether referred to as software, firmware, middleware, microcode, hardware description language, or otherwise. Machine-readable media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product. The computer-program product may comprise packaging materials.

In a hardware implementation, the machine-readable media may be part of the processing system separate from the processor. However, as those skilled in the art will readily appreciate, the machine-readable media, or any portion thereof, may be external to the processing system. By way of example, the machine-readable media may include a transmission line, a carrier wave modulated by data, and/or a computer product separate from the wireless node, all which may be accessed by the processor through the bus interface. Alternatively, or in addition, the machine-readable media, or any portion thereof, may be integrated into the processor, such as the case may be with cache and/or general register files.

The processing system may be configured as a general-purpose processing system with one or more microprocessors providing the processor functionality and external memory providing at least a portion of the machine-readable media, all linked together with other supporting circuitry through an external bus architecture. Alternatively, the processing system may be implemented with an ASIC (Application Specific Integrated Circuit) with the processor, the bus interface, the user interface in the case of an access terminal), supporting circuitry, and at least a portion of the machine-readable media integrated into a single chip, or with one or more FPGAs (Field Programmable Gate Arrays), PLDs (Programmable Logic Devices), controllers, state machines, gated logic, discrete hardware components, or any other suitable circuitry, or any combination of circuits that can perform the various functionality described throughout this disclosure. Those skilled in the art will recognize how best to implement the described functionality for the processing system depending on the particular application and the overall design constraints imposed on the overall system.

The machine-readable media may comprise a number of software modules. The software modules include instructions that, when executed by the processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module below, it will be understood that such functionality is implemented by the processor when executing instructions from that software module.

If implemented in software, the functions may be stored or transmitted over as one or more instructions or code on a computer-readable medium. Computer-readable media include both computer storage media and communication media including any medium that facilitates transfer of a computer program from one place to another. A storage medium may be any available medium that can be accessed by a computer. By way of example, and not limitation, such computer-readable media can comprise RAM, ROM, EEPROM, CD-ROM, or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared (IR), radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk, and Blu-ray® disc where disks usually reproduce data magnetically, while discs reproduce data optically with lasers. Thus, in some aspects computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media.

Thus, certain aspects may comprise a computer program product for performing the operations presented herein. For example, such a computer program product may comprise a computer-readable medium having instructions stored (and/or encoded) thereon, the instructions being executable by one or more processors to perform the operations described herein. For certain aspects, the computer program product may include packaging material.

Further, it should be appreciated that modules and/or other appropriate means for performing the methods and techniques described herein can be downloaded and/or otherwise obtained by a user terminal and/or base station as applicable. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes, and variations may be made in the arrangement, operation, and details of the methods and apparatus described above without departing from the scope of the claims.

1. An apparatus for replicating configuration information, comprising:
a receiver configured to receive a request for the configuration information used to configure the apparatus to be replicated in at least a first device;
a processing system configured to operate the apparatus based on the configuration information, wherein the operation is controlled by a second device; and
a transmitter configured to communicate the configuration information to the first device, in response to the request.

2. The apparatus of claim 1, wherein the processing system is configured to cease the operation of the apparatus after the configuration information had been communicated.

3. The apparatus of claim 1, wherein at least one of starting of the operation, stopping of the operation, monitoring progress of the operation, or managing an alarm based on the operation is controlled by the second device.

4. The apparatus of claim 1, wherein the transmitter is configured to communicate by transferring the configuration information to the first device via the second device.

5. The apparatus of claim 1, wherein the processing system is further configured to adjust the configuration information before communicating with the first device.

6. The apparatus of claim 5, wherein the configuration information is adjusted based on progress of the operation of the apparatus.

7. The apparatus of claim 1, wherein the configuration information comprises at least one of:
parameters for controlling the apparatus;
a current status of operation of the apparatus;
data logged in the apparatus; or
progress of a treatment administered by the apparatus.

8. The apparatus of claim 1, further comprising a memory configured to store the configuration information locally at the apparatus, wherein the processing system is further configured to retrieve the configuration information from the memory in response to the request.

9. The apparatus of claim 1, wherein the request is received if the apparatus is physically located close to the first device.

10. The apparatus of claim 1, wherein the first device is an infusion pump.

11. A method for replicating configuration information at an apparatus, comprising:
receiving a request for the configuration information used to configure the apparatus to be replicated in at least a first device;
operating the apparatus based on the configuration information, wherein the operation is controlled by a second device; and
communicating the configuration information to the first device, in response to the request.

12. The method of claim 11, further comprising ceasing the operation of the apparatus after the configuration information had been communicated.

13. The method of claim 11, wherein at least one of starting of the operation, stopping of the operation, monitoring progress of the operation, or managing an alarm based on the operation is controlled by the second device.

14. The method of claim 11, wherein the communicating comprises transferring the configuration information to the first device via the second device.

15. The method of claim 11, further comprising adjusting the configuration information before communicating with the first device.

16. The method of claim 15, wherein the configuration information is adjusted based on progress of the operation of the apparatus.

17. The method of claim 11, wherein the configuration information comprises at least one of:
parameters for controlling the apparatus;
a current status of operation of the apparatus;
data logged in the apparatus; or
progress of a treatment administered by the apparatus.

18. The method of claim 11, further comprising:
storing the configuration information in a memory at the apparatus; and
retrieving the configuration information from the memory in response to the request.

19. The method of claim 11, wherein the request is received if the apparatus is physically located close to the first device.

20. The method of claim 11, wherein the first device is an infusion pump.

21. An apparatus for replicating configuration information, comprising:
means for receiving a request for the configuration information used to configure the apparatus to be replicated in at least a first device;
means for operating the apparatus based on the configuration information, wherein the operation is controlled by a second device; and
means for communicating the configuration information to the first device, in response to the request.

22. The apparatus of claim 21, wherein the means for operating is configured to cease the operation of the apparatus after the configuration information had been communicated.

23. The apparatus of claim 21, wherein at least one of starting of the operation, stopping of the operation, monitoring progress of the operation, or managing an alarm based on the operation is controlled by the second device.

24. The apparatus of claim 21, wherein the means for communicating is configured to transfer the configuration information to the first device via the second device.

25. The apparatus of claim 21, further comprising means for adjusting the configuration information before communicating with the first device.

26. The apparatus of claim 25, wherein the configuration information is adjusted based on progress of the operation of the apparatus.

27. The apparatus of claim 21, wherein the configuration information comprises at least one of:
parameters for controlling the apparatus;
a current status of operation of the apparatus;
data logged in the apparatus; or
progress of a treatment administered by the apparatus.

28. The apparatus of claim 21, further comprising:
means for storing the configuration information locally at the apparatus; and
means for retrieving the configuration information from the means for storing in response to the request.

29. The apparatus of claim 21, wherein the request is received if the apparatus is physically located close to the first device.

30. The apparatus of claim 21, wherein the first device is an infusion pump.

31. A computer program product for replicating configuration information at an apparatus, comprising a computer-readable medium comprising instructions executable to:
receive a request for the configuration information used to configure the apparatus to be replicated in at least a first device;
operate the apparatus based on the configuration information, wherein the operation is controlled by a second device; and
communicate the configuration information to the first device, in response to the request.

32. An infusion pump, comprising:
a receiver configured to receive a request for configuration information used to configure the infusion pump to be replicated in at least a first device;
a pumping mechanism for administering a solution;
a processing system configured to operate the pumping mechanism based on the configuration information, wherein the operation is controlled by a second device; and
a transmitter configured to communicate the configuration information to the first device, in response to the request.

33. An apparatus for replicating configuration information, comprising:
a receiver configured to receive a request for the configuration information used to configure at least a first device to be replicated in at least a second device;
a processing system configured to control at least one of the first or second device; and
a transmitter configured to communicate the configuration information to the second device, in response to the request.

34. The apparatus of claim 33, wherein the configuration information comprises patient treatment information.

35. The apparatus of claim 33, wherein the processing system is further configured to control the at least one of the first or second device by at least one of starting an operation based on the configuration information, stopping the operation, monitoring progress of the operation, or managing an alarm based on the operation.

36. The apparatus of claim 33, wherein the processing system is further configured to obtain the configuration information from another apparatus in response to the request.

37. The apparatus of claim 33, wherein the processing system is further configured to adjust the configuration information before communicating with the second device.

38. The apparatus of claim 33, wherein the configuration information comprises at least one of:
parameters for controlling the first device;
a current status of operation of the first device;
data logged in the first device; or
progress of a treatment administered by the first device.

39. The apparatus of claim 33, wherein the transmitter is configured to communicate by transferring the configuration information to the second device via a third device.

40. The apparatus of claim 39, wherein the third device is a hub.

41. The apparatus of claim 33, wherein the receiver is further configured to receive the configuration information from the first device and wherein the processing system is further configured to:
obtain, from at least one of another apparatus or a user interface associated with the apparatus, at least one of adjustment information for, or confirmation of, the configuration information received from the first device, before communicating the configuration information to the second device; and
if the adjustment information is obtained, adjust the configuration information based on the adjustment information before the communicating, wherein the transmitter is configured to communicate the adjusted configuration information to the second device.

42. A method for replicating configuration information, comprising:
receiving a request for the configuration information used to configure at least a first device to be replicated in at least a second device;
controlling at least one of the first or second device; and
communicating the configuration information to the second device, in response to the request.

43. The method of claim 42, wherein the configuration information comprises patient treatment information.

44. The method of claim 42, wherein controlling the at least one of the first or second device comprises at least one of starting an operation based on the configuration information, stopping the operation, monitoring progress of the operation, or managing an alarm based on the operation.

45. The method of claim 42, further comprising obtaining the configuration information from an apparatus in response to the request.

46. The method of claim 42, further comprising adjusting the configuration information before communicating with the second device.

47. The method of claim 42, wherein the configuration information comprises at least one of:
parameters for controlling the first device;
a current status of operation of the first device;
data logged in the first device; or
progress of a treatment administered by the first device.

48. The method of claim 42, wherein the communicating comprises transferring the configuration information to the second device via a third device.

49. The method of claim 48, wherein the third device is a hub.

50. The method of claim 42, further comprising:
receiving the configuration information from the first device;
obtaining, from an apparatus, at least one of adjustment information for, or confirmation of, the configuration information received from the first device, before communicating the configuration information to the second device; and
if the adjustment information is obtained, adjusting the configuration information based on the adjustment information before the communicating, wherein the communicating comprises communicating the adjusted configuration information to the second device.

51. An apparatus for replicating configuration information, comprising:
means for receiving a request for the configuration information used to configure at least a first device to be replicated in at least a second device;
means for controlling at least one of the first or second device; and
means for communicating the configuration information to the second device, in response to the request.

52. The apparatus of claim 51, wherein the configuration information comprises patient treatment information.

53. The apparatus of claim 51, wherein the means for controlling the at least one of the first or second device is configured to at least one of start an operation based on the configuration information, stop the operation, monitor progress of the operation, or manage an alarm based on the operation.

54. The apparatus of claim 51, further comprising means for obtaining the configuration information from another apparatus in response to the request.

55. The apparatus of claim 51, further comprising means for adjusting the configuration information before communicating with the second device.

56. The apparatus of claim 51, wherein the configuration information comprises at least one of:
parameters for controlling the first device;
a current status of operation of the first device;
data logged in the first device; or
progress of a treatment administered by the first device.

57. The apparatus of claim 51, wherein the means for communicating is configured to transfer the configuration information to the second device via a third device.

58. The apparatus of claim 57, wherein the third device is a hub.

59. The apparatus of claim 51, further comprising:
means for receiving the configuration information from the first device;
means for obtaining, from at least one of another apparatus or a user interface associated with the apparatus, at least one of adjustment information for, or confirmation of, the configuration information received from the first device, before communicating the configuration information to the second device; and
means for adjusting, if the adjustment information is obtained, the configuration information based on the adjustment information before the communicating, wherein the means for communicating is configured to communicate the adjusted configuration information to the second device.

60. A computer program product for replicating configuration information, comprising a computer-readable medium comprising instructions executable to:
receive a request for the configuration information used to configure at least a first device to be replicated in at least a second device;
control at least one of the first or second device; and
communicate the configuration information to the second device, in response to the request.

61. A hub for replicating configuration information, comprising:
at least one antenna;
a receiver configured to receive, via the at least one antenna, a request for the configuration information used to configure at least a first device to be replicated in at least a second device;
a processing system configured to control at least one of the first or second device; and
a transmitter configured to communicate, via the at least one antenna, the configuration information to the second device, in response to the request.

62. A first apparatus for replicating configuration information, comprising:
a receiver configured to receive, from a second apparatus, the configuration information used by the second apparatus to configure at least one first device; and
a processing system configured to control at least one second device based on the configuration information.

63. The first apparatus of claim 62, wherein the at least one second device is the at least one first device.

64. The first apparatus of claim 62, further comprising a transmitter configured to communicate the configuration information to the at least one second device.

65. The first apparatus of claim 64, wherein the processing system is further configured to adjust the configuration information before communicating with the at least one second device.

66. The first apparatus of claim 64, wherein the processing system is further configured to:
obtain, from at least one of a third apparatus or a user interface associated with the first apparatus, at least one of adjustment information for, or confirmation of, the configuration information received from the second apparatus, before communicating the configuration information to the at least one second device; and
if the adjustment information is obtained, adjust the configuration information based on the adjustment information before the communicating, wherein the transmitter is configured to communicate the adjusted configuration information to the at least one second device.

67. The first apparatus of claim 62, wherein the configuration information comprises at least one of:
parameters for controlling the at least one first device;
a current status of operation of the at least one first device;
data logged in the at least one first device; or
progress of a treatment administered by the at least one first device.

68. The first apparatus of claim 62, wherein the processing system is configured to control the at least one second device by at least one of starting an operation based on the configuration information, stopping the operation, monitoring progress of the operation, or managing an alarm based on the operation.

69. A method for replicating configuration information at a first apparatus, comprising:
receiving, from a second apparatus, the configuration information used by the second apparatus to configure at least one first device; and
controlling at least one second device based on the configuration information.

70. The method of claim 69, wherein the at least one second device is the at least one first device.

71. The method of claim 69, further comprising communicating the configuration information to the at least one second device.

72. The method of claim 71, further comprising adjusting the configuration information before communicating with the at least one second device.

73. The method of claim 71, further comprising:
obtaining, from at least one of a third apparatus or a user interface associated with the first apparatus, at least one of adjustment information for, or confirmation of, the configuration information received from the second apparatus, before communicating the configuration information to the at least one second device; and
if the adjustment information is obtained, adjusting the configuration information based on the adjustment information before the communicating, wherein the communicating comprises communicating the adjusted configuration information to the at least one second device.

74. The method of claim 69, wherein the configuration information comprises at least one of:
parameters for controlling the at least one first device;
a current status of operation of the at least one first device;
data logged in the at least one first device; or
progress of a treatment administered by the at least one first device.

75. The method of claim 69, wherein controlling the at least one second device comprises at least one of starting an operation based on the configuration information, stopping the operation, monitoring progress of the operation, or managing an alarm based on the operation.

76. A first apparatus for replicating configuration information, comprising:
means for receiving, from a second apparatus, the configuration information used by the second apparatus to configure at least one first device; and
means for controlling at least one second device based on the configuration information.

77. The first apparatus of claim 76, wherein the at least one second device is the at least one first device.

78. The first apparatus of claim 76, further comprising means for communicating the configuration information to the at least one second device.

79. The first apparatus of claim 78, further comprising means for adjusting the configuration information before communicating with the at least one second device.

80. The first apparatus of claim 78, further comprising:
means for obtaining, from at least one of a third apparatus or a user interface associated with the first apparatus, at least one of adjustment information for, or confirmation of, the configuration information received from the second apparatus, before communicating the configuration information to the at least one second device; and
means for adjusting, if the adjustment information is obtained, the configuration information based on the adjustment information before the communicating, wherein the means for communicating is configured to communicate the adjusted configuration information to the at least one second device.

81. The first apparatus of claim 76, wherein the configuration information comprises at least one of:
parameters for controlling the at least one first device;
a current status of operation of the at least one first device;
data logged in the at least one first device; or
progress of a treatment administered by the at least one first device.

82. The first apparatus of claim 76, wherein the means for controlling the at least one second device is configured to at least one of start an operation based on the configuration information, stop the operation, monitor progress of the operation, or manage an alarm based on the operation.

83. A computer program product for replicating configuration information at a first apparatus, comprising a computer-readable medium comprising instructions executable to:
receive, from a second apparatus, the configuration information used by the second apparatus to configure at least one first device; and
control at least one second device based on the configuration information.

84. A hub for replicating configuration information, comprising:
at least one antenna;
a receiver configured to receive, via the at least one antenna from an apparatus, the configuration information used by the apparatus to configure at least one first device; and
a processing system configured to control at least one second device based on the configuration information.
